(19) United States
(12) Patent Application Publication
(10) Pub. No.: US 2018/0338813 A1
(43) Pub. Date: Nov. 29, 2018

(54) MEDICAL OBSERVATION APPARATUS AND ZOOM CONTROL METHOD

(71) Applicant: Sony Olympus Medical Solutions Inc., Tokyo (JP)

(72) Inventor: Hidenori TAGUCHI, Kanagawa (JP)

(73) Assignee: Sony Olympus Medical Solutions Inc., Tokyo (JP)

(21) Appl. No.: 15/961,934

(22) Filed: Apr. 25, 2018

(30) Foreign Application Priority Data
May 26, 2017 (JP) 2017-104175

(51) Int. Cl. A61B 90/00; A61B 90/25; H04N 5/232

(52) U.S. Cl. CPC A61B 90/361; A61B 90/50; H04N 5/23296; A61B 90/25

(57) ABSTRACT

There is provided a medical observation apparatus including: an imaging control section that controls a zoom function of an imaging device on a basis of a first toggle operation that toggles from a mode that controls an optical zoom to a mode that controls an electronic zoom. The imaging control section controls the zoom function so that the electronic zoom is executed on a predetermined region in a medical captured image captured by the imaging device, at an optical magnification of an optical system of the imaging device corresponding to when the first toggle operation was detected.

### CROSS REFERENCE TO RELATED APPLICATIONS

This application claims the benefit of Japanese Priority Patent Application JP 2017-104175 filed May 26, 2017, the entire contents of which are incorporated herein by reference.

### BACKGROUND

The present disclosure relates to a medical observation apparatus and a zoom control method.

Recently, in the medical field, to support microsurgery such as neurosurgical procedures, for example, medical observation apparatus capable of enlarged observation of an observation target such as an affected area are used in some cases. Examples of medical observation apparatus include a medical observation apparatus provided with an optical microscope, and a medical observation apparatus provided with an imaging device that functions as an electronic imaging microscope. In the following, the above medical observation apparatus provided with an optical microscope will be designated an “optical medical observation apparatus”. Also, in the following, the above medical observation apparatus provided with an imaging device will be designated an “electronic imaging medical observation apparatus” or simply a “medical observation apparatus” in some cases.

Also, technology related to an electronic imaging medical observation apparatus capable of recognizing a treatment tool used in a medical procedure, and adjusting an observation magnification while also moving the position of an imaging device on the basis of the recognition result, is being developed. Examples of the above technologies include the technology described in JP 2006-26229A below.

### SUMMARY

In an optical medical observation apparatus, the zoom magnification of an image is changed by an optical zoom. Herein, an optical zoom is a zoom method that changes the zoom magnification by moving a zoom lens included in the optical system to change the focal length of the zoom lens. Thus, when the zoom magnification is changed in the optical medical observation apparatus, the zoom magnification only changes continuously. Also, with optical zoom, since it is necessary to physically move the zoom lens, it takes approximately several seconds to more than ten seconds from the start to the completion of the optical zoom.

Also, in the case in which an optical medical observation apparatus is used in surgery, a surgeon changes the zoom magnification as appropriate to obtain a desired surgical field. Thus, in the case in which an optical medical observation apparatus is used in surgery, since it takes approximately several seconds to more than ten seconds until the desired surgical field is obtained every time the zoom magnification is changed, there is a risk of lowered surgical efficiency.

For example, the medical observation apparatus in which the technology described in JP 2006-26229A is used automatically adjusts the imaging device position and the observation magnification on the basis of a recognized treatment tool (corresponding to the surgical instrument described in JP 2006-26229A). Thus, through the use of the technology described in JP 2006-26229A, for example, it is possible to improve convenience for the surgeon or the like who performs a medical procedure using the medical observation apparatus. However, since an optical zoom with an automatically adjusting observation magnification is likewise used in a medical observation apparatus in which the technology described in JP 2006-26229A is used, it takes time until the surgical field desired by the surgeon is obtained.

The present disclosure proposes a novel and improved medical observation apparatus and zoom control method capable of potentially shortening the time taken for zooming of a medical captured image.

According to an embodiment of the present disclosure, there is provided a medical observation apparatus including: an imaging control section that controls a zoom function of an imaging device on a basis of a first toggle operation that toggles from a mode that controls an optical zoom to a mode that controls an electronic zoom. The imaging control section controls the zoom function so that the electronic zoom is executed on a predetermined region in a medical captured image captured by the imaging device, at an optical magnification of an optical system of the imaging device corresponding to when the first toggle operation was detected.

In addition, according to an embodiment of the present disclosure, there is provided a zoom control method executed by a medical observation apparatus, the method including: controlling a zoom function of an imaging device on a basis of a first toggle operation that toggles from a mode that controls an optical zoom to a mode that controls an electronic zoom. In the controlling, the zoom function is controlled so that the electronic zoom is executed on a predetermined region in a medical captured image captured by the imaging device, at an optical magnification of an optical system of the imaging device corresponding to when the first toggle operation was detected.

According to an embodiment of the present disclosure, the time taken for zooming of a medical captured image is potentially shortened.

Note that the effects described above are not necessarily limitative. With or in the place of the above effects, there may be achieved any one of the effects described in this specification or other effects that may be grasped from this specification.

### BRIEF DESCRIPTION OF THE DRAWINGS

FIG. 1 is an explanatory diagram illustrating an example of a configuration of a medical observation system according to the present embodiment;

FIG. 2 is an explanatory diagram illustrating an example of a use case in which the medical observation system according to the present embodiment is used;

FIG. 3 is an explanatory diagram illustrating another example of a use case in which the medical observation system according to the present embodiment is used;

FIG. 4 is an explanatory diagram for explaining an example of the configuration of an imaging device provided in a medical observation apparatus according to the present embodiment;

FIG. 5 is a function block diagram illustrating an example of a configuration of a medical observation apparatus according to the present embodiment;

FIG. 6 is an explanatory diagram for describing an example of a process related to the zoom control method according to the present embodiment;

FIG. 7 is an explanatory diagram for describing an example of a process related to the zoom control method according to the present embodiment;

FIG. 8 is an explanatory diagram for describing another example of a process related to a zoom control method according to the present embodiment; and

FIG. 9 is a flowchart illustrating an example of a process related to the zoom control method according to the present embodiment.

### DETAILED DESCRIPTION OF THE EMBODIMENT(S)

Hereinafter, (a) preferred embodiment(s) of the present disclosure will be described in detail with reference to the appended drawings. In this specification and the appended drawings, structural elements that have substantially the same function and structure are denoted with the same reference numerals, and repeated explanation of these structural elements is omitted.

The description hereinafter will proceed in the following order.

1. Medical observation system according to present embodiment and zoom control method according to present embodiment

2. Medical sterile cover according to present embodiment

### (Medical Observation System According to Present Embodiment and Zoom Control Method According to Present Embodiment)

Hereinafter, an example of a medical observation system according to the present embodiment will be described, while the zoom control method according to the present embodiment will also be described.

### [1] Configuration of Medical Observation System

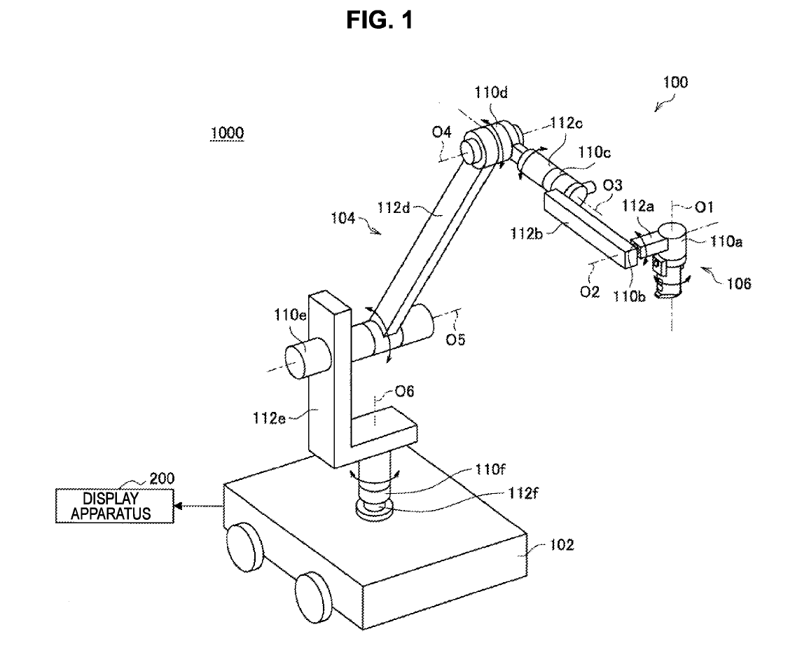

FIG. 1 is an explanatory diagram illustrating an example of the configuration of a medical observation system 1000 according to the present embodiment. The medical observation system 1000 includes a medical observation apparatus 100 and a display apparatus 200, for example.

Note that the medical observation system according to the present embodiment is not limited to the example illustrated in FIG. 1.

For example, the medical observation system according to the present embodiment additionally may include a control apparatus (not illustrated) that controls various operations in the medical observation apparatus 100. In the medical observation system 1000 illustrated in FIG. 1, as described later, an example is illustrated in which, by providing the medical observation apparatus 100 with a control section (described later) that performs processes according to the zoom control method according to the present embodiment, the medical observation apparatus 100 includes the functions of the control apparatus (not illustrated).

Examples of the control apparatus (not illustrated) include arbitrary equipment capable of performing processes according to the zoom control method according to the present embodiment, such as a “medical controller” and a “computer such as a server”. Also, the control apparatus (not illustrated) may be, for example, an integrated circuit (IC) that can be embedded in equipment like the above.

Additionally, the medical observation system according to the present embodiment may also be a configuration that includes multiple medical observation apparatuses 100 and display apparatuses 200. In the case of including multiple medical observation apparatuses 100, in each medical observation apparatus 100, processes according to the zoom control method in the medical observation apparatus 100 described later are performed. Also, in the case in which the medical observation system according to the present embodiment is a configuration that includes multiple medical observation apparatuses 100 and display apparatuses 200, the medical observation apparatus 100 and the display apparatus 200 may be associated in a 1-to-1 manner, or multiple medical observation apparatuses 100 may be associated with a single display apparatus 200. In the case in which multiple medical observation apparatuses 100 are associated with a single display apparatus 200, which medical observation apparatus 100 provides a taken image to be displayed on a display screen is switched by performing a switching operation or the like in the display apparatus 200, for example.

Additionally, the medical observation system according to the present embodiment may also include a gaze detection sensor capable of detecting gaze. Examples of a sensor functioning as a gaze detection sensor include “a sensor unit including a stereo camera and a processor that detects gaze from a captured image captured by the stereo camera”. Also, the gaze detection sensor may be a sensor of arbitrary method capable of detecting gaze, such as a configuration that detects gaze with infrared rays, for example. In the case in which the medical observation system according to the present embodiment includes a gaze detection sensor, the gaze detection sensor is provided at a position enabling the detection of the position of the gaze on the display screen of the display apparatus 200, for example.

In the case in which the medical observation system according includes a gaze detection sensor, as described later, the medical observation apparatus 100 utilizes a detection result of the gaze detection sensor to execute a process related to the zoom control method according to the present embodiment.

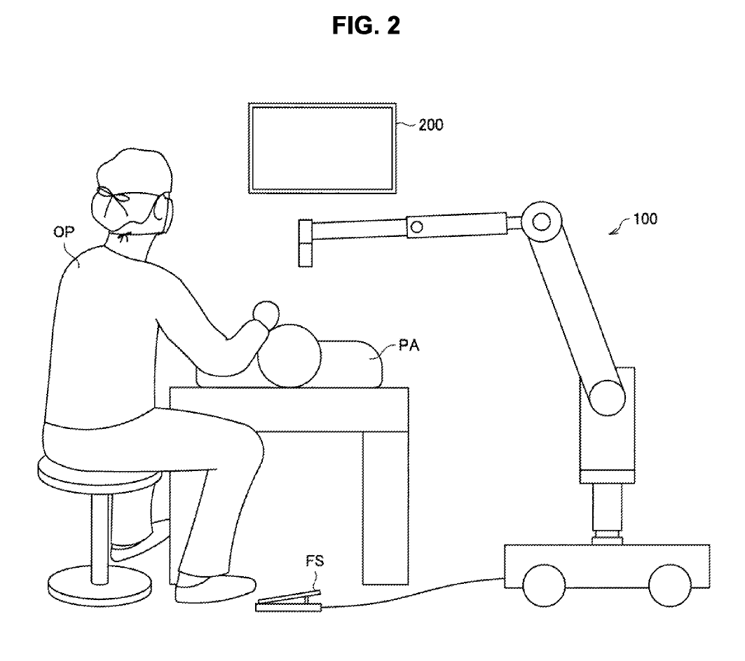

FIG. 2 is an explanatory diagram illustrating an example of a use case in which the medical observation system 1000 according to the present embodiment is used.

By an imaging device (described later) provided in the medical observation apparatus 100, an observation target patient PA (a patient who undergoes a medical procedure) is imaged. In the following, a captured image captured by the medical observation apparatus according to the present embodiment, such as a captured image that captures the above patient who undergoes a medical procedure, is designated a “medical captured image”.

The medical captured image captured in the medical observation apparatus 100 is displayed on a display screen of a display apparatus 200. Subsequently, a surgeon OP (an example of a user of the medical observation apparatus 100) who performs a medical procedure by using the medical observation apparatus 100 performs the medical procedure on the patient PA while looking at the medical captured image displayed on the display screen of the display apparatus 200.

Also, the surgeon OP operates an operating device external to the medical observation apparatus 100, such as a footswitch FS, or an operating device (described later) provided in the medical observation apparatus 100, thereby causing an arm (described later) and the imaging device (described later) provided in the medical observation apparatus 100 to operate, and putting the medical observation apparatus 100 into a desired state.

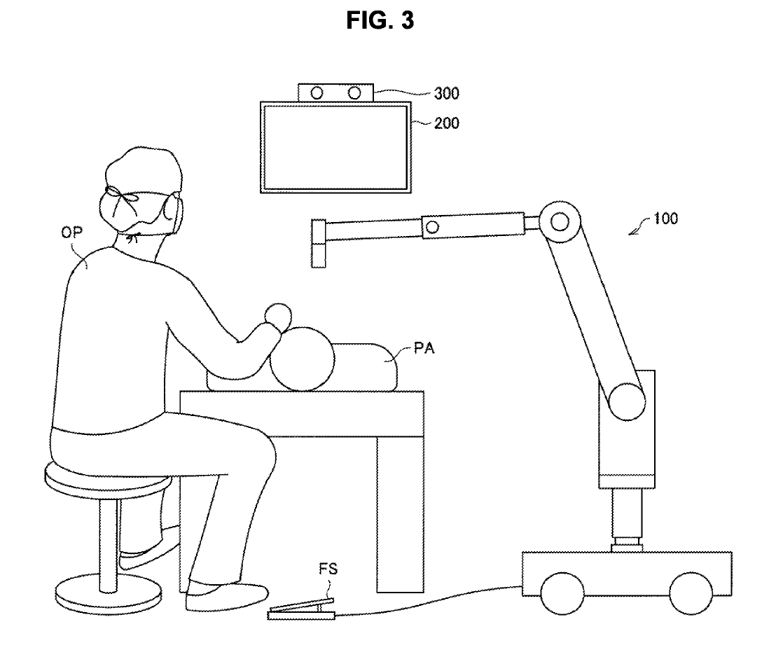

FIG. 3 is an explanatory diagram illustrating another example of a use case in which the medical observation system 1000 according to the present embodiment is used. FIG. 3 illustrates a use case for a case in which the medical observation system 1000 additionally includes a gaze detection sensor 300.

The gaze detection sensor 300 is provided on the upper side of the display screen of the display apparatus 200 as illustrated in FIG. 3, for example, and detects gaze with respect to the display screen of the display apparatus 200. Additionally, the gaze detection sensor 300 outputs information indicating a gaze detection result.

The detection target whose gaze is detected by the gaze detection sensor 300 may be an unspecified person, or a specific person such as the surgeon. In the case in which the gaze detection sensor 300 treats a specific person as the detection target, the gaze detection sensor 300 specifies the detection target by executing a facial detection process that detects a set face from a captured image captured by the stereo camera, for example. Subsequently, the gaze detection sensor 300 detects the gaze of the specified detection target, and outputs information indicating the gaze detection result.

Examples of the information indicating the gaze detection result include data indicating the position of gaze on the display screen of the display apparatus 200 (for example, planar coordinate data expressed by treating an arbitrary point, such as the upper-left of the display screen of the display apparatus 200, as the origin). For example, the gaze detection sensor 300 finds the position of an intersection between a gaze vector corresponding to the detected gaze and a set plane corresponding to the display screen of the display apparatus 200. In the case in which the position of the above intersection is found, the gaze detection sensor 300 treats the computed position of the intersection as the position of the gaze on the display screen of the display apparatus 200, and outputs data indicating the position of the gaze on the display screen of the display apparatus 200 as the information indicating the gaze detection result.

In addition, the information indicating the gaze detection result may also be data (such as flag data, for example) indicating that the gaze does not lie on the display screen of the display apparatus 200. In the case in which the position of the above intersection is not found, the gaze detection sensor 300 outputs data indicating that the gaze does not lie on the display screen of the display apparatus 200 as the information indicating the gaze detection result.

Note that the method of specifying the position of the gaze on the display screen of the display apparatus 200 obviously is not limited to the example indicated above. Also, the process of specifying the position of the gaze on the display screen of the display apparatus 200 may also be executed by an apparatus external to the gaze detection sensor 300 (such as the medical observation apparatus 100 or a control apparatus (not illustrated), for example).

In the case in which the medical observation system 1000 includes the gaze detection sensor 300, the gaze of the surgeon OP is detected by the gaze detection sensor 300, for example. The medical observation apparatus 100 executes a process related to the zoom control method described later on the basis of the information indicating the gaze detection result output from the gaze detection sensor 300, and controls the zoom function of the imaging device (described later). In other words, in the case in which the medical observation system 1000 additionally includes the gaze detection sensor 300, the surgeon OP is able to change the zoom magnification of the imaging device (described later) with one's gaze.

Hereinafter, each apparatus included in the medical observation system 1000 will be described.

### [1-1] Display Apparatus 200

The display apparatus 200 is a display device in the medical observation system 1000, and corresponds to an external display device from the perspective of the medical observation apparatus 100. The display apparatus 200 displays various images on a display screen, such as a medical captured image (a moving image or multiple still images; the same applies hereinafter) taken in the medical observation apparatus 100, or an image related to a user interface (UI), for example. In addition, the display apparatus 200 may also be a configuration capable of 3D display. The display on the display apparatus 200 is controlled by, for example, the medical observation apparatus 100 or the control apparatus (not illustrated).

In the medical observation system 1000, the display apparatus 200 is installed in an arbitrary location visible to a person involved in a surgery inside an operating room, such as on a wall, the ceiling, or the floor of the operating room, for example. Examples of the display apparatus 200 include a liquid crystal display, an organic electro-luminescence (EL) display, a cathode ray tube (CRT) display, and the like.

Note that the display apparatus 200 is not limited to the example illustrated above.

For example, the display apparatus 200 may also be an arbitrary wearable apparatus that is used by being worn on the body of the surgeon or the like, such as a head-mounted display, an eyewear-type apparatus, or the like.

The display apparatus 200 runs on electric power supplied from an internal power source such as a battery provided in the display apparatus 200, on electric power supplied from a connected external power source, or the like, for example.

### [1-2] Medical Observation Apparatus 100

The medical observation apparatus 100 is an electronic imaging medical observation apparatus. For example, in the case in which the medical observation apparatus 100 is used during surgery, the surgeon (one example of the user of the medical observation apparatus 100) observes an operating site while referring to a medical captured image which has been taken by the medical observation apparatus 100 and displayed on the display screen of the display apparatus 200, and performs various treatments, such as techniques depending on the surgical procedure, on the operating site.

First, FIG. 1 will be referenced to describe an example of a hardware configuration of the medical observation apparatus 100.

The medical observation apparatus 100 is provided with a base 102, an arm 104, and an imaging device 106, for example.

Additionally, although not illustrated in FIG. 1, the medical observation apparatus 100 may also be provided with, for example, one or multiple processors (not illustrated) including a computational circuit such as a microprocessing unit (MPU), read-only memory (ROM; not illustrated), random access memory (RAM; not illustrated), a recording medium (not illustrated), and a communication device (not illustrated). The medical observation apparatus 100 runs on electric power supplied from an internal power source such as a battery provided in the medical observation apparatus 100, on electric power supplied from a connected external power source, or the like, for example.

The one or multiple processors (not illustrated) function as the control section described later. The ROM (not illustrated) stores programs and control data such as computational parameters used by the one or multiple processors (not illustrated). The RAM (not illustrated) temporarily stores programs executed by the one or multiple processors (not illustrated), or the like.

The recording medium (not illustrated) functions as a storage section. A variety of data is stored on the recording medium (not illustrated), including data related to the zoom control method according to the present embodiment, for example. Herein, examples of the recording medium (not illustrated) include a magnetic recording medium such as a hard disk, non-volatile memory such as flash memory, and the like. Additionally, the recording medium (not illustrated) may also be removable from the medical observation apparatus 100.

The communication device (not illustrated) is a communication device provided in the medical observation apparatus 100, and fulfills a role of communicating in a wireless or wired manner with an external apparatus such as the display apparatus 200. Herein, examples of the communication device (not illustrated) include an IEEE 802.15.1 port and transmitting-receiving circuit (wireless communication), an IEEE 802.11 port and transmitting-receiving circuit (wireless communication), a communication antenna and a radio frequency (RF) circuit (wireless communication), a local area network (LAN) terminal and a transmitting-receiving circuit (wired communication), and the like. The communication device (not illustrated) may also be a configuration capable of communicating with one or multiple external apparatus by multiple communication methods.

### [1-2-1] Base 102

The base 102 is the base of the medical observation apparatus 100. One end of the arm 104 is connected to the base 102, and the base 102 supports the arm 104 and the imaging device 106.

Also, casters are provided on the base 102, for example, and the medical observation apparatus 100 contacts the floor through the casters. By providing the casters, the medical observation apparatus 100 is able to move easily over the floor by the casters.

### [1-2-2] Arm 104

The arm 104 includes multiple links joined to each other by joint sections. In addition, the arm 104 supports the imaging device 106. The imaging device 106 supported by the arm 104 is movable three-dimensionally, and after moving, the position and the attitude of the imaging device 106 are maintained by the arm 104.

More specifically, the arm 104 includes, for example, multiple joint sections 110a, 110b, 110c, 110d, 110e, and 110f, and multiple links 112a, 112b, 112c, 112d, 112e, and 112f rotatably joined to each other by the joint sections 110a, 110b, 110c, 110d, 110e, and 110f. The rotatable range of each of the joint sections 110a, 110b, 110c, 110d, 110e, and 110f is set arbitrarily during the design stage, the manufacturing stage, or the like so that the desired motion of the arm 104 is realized.

In other words, in the medical observation apparatus 100 illustrated in FIG. 1, six degrees of freedom are realized in relation to the movement of the imaging device 106 by six rotation axes (first axis O1, second axis O2, third axis O3, fourth axis O4, fifth axis O5, and sixth axis O6) corresponding to the six joint sections 110a, 110b, 110c, 110d, 110e, and 110f included in the arm 104. More specifically, in the medical observation apparatus 100 illustrated in FIG. 1, motion with six degrees of freedom, including three degrees of translational freedom and three degrees of rotational freedom, is realized.

Actuators (not illustrated) are provided in each of the joint sections 110a, 110b, 110c, 110d, 110e, and 110f. Each of the joint sections 110a, 110b, 110c, 110d, 110e, and 110f rotates about the corresponding rotation axis by the driving of the actuators (not illustrated). The driving of the actuators (not illustrated) is controlled by, for example, a processor that functions as the control section described later, or an external control apparatus (not illustrated).

By having each of the joint sections 110a, 110b, 110c, 110d, 110e, 110f rotate about the corresponding rotation axis by the driving of the actuators (not illustrated), various operations of the arm 104, such as extending and contracting (folding up) the arm 104, for example, are realized.

The joint section 110a has an approximately cylindrical shape, and supports the imaging device 106 (the top end of the imaging device 106 in FIG. 1) on the front end portion of the joint section 110a (the bottom end portion in FIG. 1), so as to allow revolution about a rotation axis (first axis O1) parallel to the central axis of the imaging device 106. Herein, the medical observation apparatus 100 is configured so that the first axis O1 is aligned with the optical axis in the imaging device 106. In other words, by causing the imaging device 106 to revolve about the first axis O1 illustrated in FIG. 1, the medical captured image captured by the imaging device 106 becomes an image which has changed so that the field of view rotates.

The link 112a is an approximately rod-shaped member, and securely supports the joint section 110a. The link 112a extends in a direction orthogonal to the first axis O1, for example, and is connected to the joint section 110b.

The joint section 110b has an approximately cylindrical shape, and supports the link 112a so as to allow revolution about a rotation axis (second axis O2) orthogonal to the first axis O1. Also, the link 112b is securely connected to the joint section 110b.

The link 112b is an approximately rod-shaped member, and extends in a direction orthogonal to the second axis O2. Also, each of the joint section 110b and the joint section 110c is connected to the link 112b.

The joint section 110c has an approximately cylindrical shape, and supports the link 112b so as to allow revolution about a rotation axis (third axis O3) mutually orthogonal to each of the first axis O1 and the second axis O2. Also, one end of the link 112c is securely connected to the joint section 110c.

Herein, by having the front end side (the side on which the imaging device 106 is provided) of the arm 104 revolve about the second axis O2 and the third axis O3, the imaging device 106 can be made to move so that the position of the imaging device 106 in the horizontal plane is changed. In other words, in the medical observation apparatus 100, controlling the rotation about the second axis O2 and the third axis O3 makes it possible to move the field of view of the medical captured image in a flat plane.

The link 112c is a member in which one end has an approximately cylindrical shape, and the other end has an approximately rod-like shape. On the side of the one end of the link 112c, the joint section 110c is securely connected so that the central axis of the joint section 110c and the central axis of the approximately cylindrical shape are the same. Also, on the side of the other end of the link 112c, the joint section 110d is connected.

The joint section 110d has an approximately cylindrical shape, and supports the link 112c so as to allow revolution about a rotation axis (fourth axis O4) orthogonal to the third axis O3. The link 112d is securely connected to the joint section 110d.

The link 112d is an approximately rod-shaped member, and extends orthogonally to the fourth axis O4. One end of the link 112d is securely connected to the joint section 110d so as to abut the approximately cylindrical side face of the joint section 110d. Also, the joint section 110e is connected to the other end of the link 112d (the end on the opposite side of the side where the joint section 110d is connected).

The joint section 110e has an approximately cylindrical shape, and supports one end of the link 112d so as to allow revolution about a rotation axis (fifth axis O5) parallel to the fourth axis O4. Also, one end of the link 112e is securely connected to the joint section 110e.

Herein, the fourth axis O4 and the fifth axis O5 are rotation axis about which the imaging device 106 may be moved in the vertical direction. By having the front end side (the side on which the imaging device 106 is provided) of the arm 104 revolve about the fourth axis O4 and the fifth axis O5, the position of the imaging device 106 in the vertical direction changes. Thus, by having the front end side (the side on which the imaging device 106 is provided) of the arm 104 revolve about the fourth axis O4 and the fifth axis O5, changing the distance between the imaging device 106 and an observation target, such as an operating site of a patient, becomes possible.

The link 112e is a member that includes a combination of a first member having an approximate L-shape with one edge extending in the vertical direction while the other edge extends in the horizontal direction, and a rod-like second member that extends vertically downward from the part of the first member that extends in the horizontal direction. The joint section 110e is securely connected to the part of the first member of the link 112e that extends in the vertical direction. Also, the joint section 110f is connected to the second member of the link 112e.

The joint section 110f has an approximately cylindrical shape, and supports the link 112e so as to allow revolution about a rotation axis (sixth axis O6) parallel to the vertical direction. Also, the link 112f is securely connected to the joint section 110f.

The link 112f is an approximately rod-shaped member, and extends in the vertical direction. The joint section 110f is connected to one end of the link 112f. Also, the other end of the link 112f (the end on the opposite side of the side where the joint section 110f is connected) is securely connected to the base 102.

By having the arm 104 include the configuration indicated above, in the medical observation apparatus 100, six degrees of freedom are realized with respect to the movement of the imaging device 106.

Note that the configuration of the arm 104 is not limited to the example indicated above.

For example, each of the joint sections 110a, 110b, 110c, 110d, 110e, and 110f of the arm 104 may be provided with a brake that restrains rotation in each of the joint sections 110a, 110b, 110c, 110d, 110e, and 110f. The brake according to the present embodiment may be a brake of an arbitrary method, such as a mechanically driven brake or an electrically driven electromagnetic brake, for example.

The driving of the above brakes is controlled by, for example, a processor that functions as the control section described later, or an external control apparatus (not illustrated). By controlling the driving of the above brakes, in the medical observation apparatus 100, the operating mode of the arm 104 is set. Examples of operating modes of the arm 104 include a locked mode and a free mode.

Herein, the locked mode according to the present embodiment is, for example, an operating mode in which the position and the attitude of the imaging device 106 are locked by using brakes to restrain rotation about each rotation axis provided in the arm 104. By having the arm 104 enter the locked mode, the operating state of the medical observation apparatus 100 becomes a locked state in which the position and the attitude of the imaging device 106 are locked.

Also, the free mode according to the present embodiment is an operating mode in which the above brakes are released, thereby allowing each rotation axis provided in the arm 104 to rotate freely. For example, in the free mode, the position and the attitude of the imaging device 106 are adjustable by direct operations performed by the surgeon. Herein, a direct operation according to the present embodiment means, for example, an operation in which the surgeon grips the imaging device 106 with his or her hand, and directly moves the imaging device 106.

### [1-2-3] Imaging Device 106

The imaging device 106 is supported by the arm 104, and images an observation target such as an operating site of a patient, for example. Imaging in the imaging device 106 is controlled by, for example, a processor that functions as the control section described later, or an external control apparatus (not illustrated).

The imaging device 106 has a configuration corresponding to an electronic imaging microscope, for example.

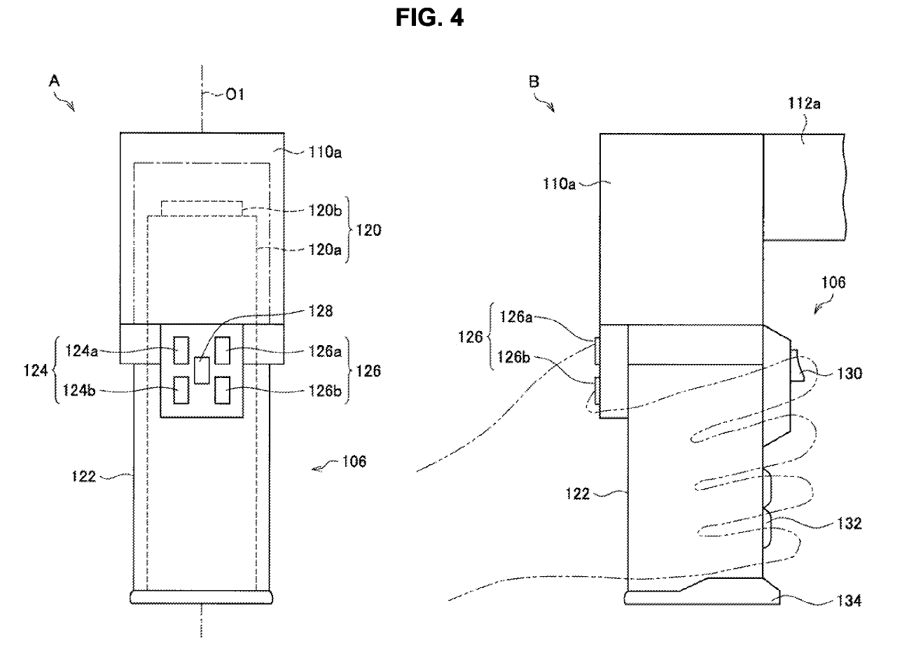

FIG. 4 is an explanatory diagram for explaining an example of the configuration of the imaging device 106 provided in the medical observation apparatus 100 according to the present embodiment.

For example, the imaging device 106 includes an imaging member 120 and a barrel member 122 having an approximately cylindrical shape, with the imaging member 120 being provided inside the barrel member 122.

On an aperture on the bottom end of the barrel member 122 (the lower end in FIG. 4), for example, a cover glass (not illustrated) for protecting the imaging member 120 is provided.

Additionally, for example, a light source (not illustrated) is provided inside the barrel unit 122, and during imaging, the subject is irradiated with illuminating light radiating from the light source through the cover glass. Reflected light (observation light) from the subject irradiated with illuminating light enters the imaging member 120 through the cover glass (not illustrated), whereby an image signal indicating the subject (an image signal indicating a taken image) is obtained by the imaging member 120.

As the imaging member 120, any of various known types of configurations used in an electronic imaging microscope section can be applied.

To give one example, the imaging member 120 includes an optical system 120a and an image sensor 120b including an imaging element that takes an image of an observation target with light transmitted through the optical system 120a, for example. The optical system 120a includes optical elements such as a mirror and one or multiple lenses, such as an objective lens, a zoom lens, and a focus lens, for example. Examples of the image sensor 120b include an image sensor using multiple imaging elements, such as a complementary metal-oxide semiconductor (CMOS) or a charge-coupled device (CCD).

The imaging member 120 may also include a pair of imaging elements, or in other words, be configured to function as what is called a stereo camera. The imaging member 120 is equipped with one or multiple functions typically provided in an electronic imaging microscope section, including at least a zoom function (an optical zoom function and an electronic zoom function), such as an autofocus (AF) function.

In addition, the imaging member 120 may also be configured to be capable of imaging at what are called high resolutions, such as 4K and 8K, for example. By configuring the imaging member 120 to be capable of imaging at high resolutions, it becomes possible to ensure a predetermined resolution (such as full HD image quality, for example), while also displaying an image on the display apparatus 200 having a large display screen, such as 50 inches or more, for example. For this reason, visibility is improved for the surgeon watching the display screen. Also, by configuring the imaging member 120 to be capable of imaging at high resolutions, even if the taken image is enlarged by the electronic zoom function and displayed on the display screen of the display apparatus 200, it is still possible to ensure a predetermined resolution. Furthermore, in the case of using the electronic zoom function to ensure a predetermined resolution, since it is possible to reduce the performance of the optical zoom function in the imaging device 106, the optical system of the imaging device 106 can be simplified, and the imaging device 106 can be configured more compactly.

In the imaging device 106, for example, various operating devices for controlling the operation of the imaging device 106 are provided. For example, in FIG. 4, a zoom switch 124, a focus switch 126, an electronic zoom toggle switch 128, and an operating mode change switch 130 are provided on the imaging device 106. Note that the positions and shapes in which to provide the zoom switch 124, the focus switch 126, the electronic zoom toggle switch 128, and the operating mode change switch 130 obviously are not limited to the example illustrated in FIG. 4.

The zoom switch 124, the focus switch 126, and the electronic zoom toggle switch 128 are an example of an operating device for adjusting the imaging parameters in the imaging device 106.

The zoom switch 124 causes the optical zoom included in the imaging device 106 to work, and is an example of an operating device for changing the zoom magnification by optical zoom. The zoom switch 124 includes, for example, a zoom-in switch 124a that increases the zoom magnification (enlargement ratio) by optical zoom, and a zoom-out switch 124b that decreases the zoom magnification by optical zoom. By performing an operation on the zoom switch 124, the zoom lens included in the optical system 120a moves, the zoom magnification is adjusted, and the zoom is adjusted. The operation on the zoom switch 124 is an operation for changing the zoom magnification by optical zoom. Hereinafter, an operation for changing the zoom magnification by optical zoom, like an operation on the zoom switch 124, is designated an “optical zoom operation”.

The focus switch 126 causes the focus function included in the imaging device 106 to work, and is an example of an operating device for adjusting the focus. The focus switch 126 includes, for example, a long-range focus switch 126a that increases the focal length to the observation target (subject), and a close-range focus switch 126b that decreases the focal length to the observation target. By performing an operation on the focus switch 126, the focal length is adjusted, and the focus is adjusted.

The electronic zoom toggle switch 128 is an example of an operating device for toggling the zoom function included in the imaging device 106. By performing an operation on the electronic zoom toggle switch 128, a mode for controlling the zoom function of the imaging device 106 is toggled from a mode that controls the optical zoom to a mode that controls the electronic zoom. In other words, an operation on the electronic zoom toggle switch 128 corresponds to a toggle operation that toggles from the mode that controls the optical zoom to the mode that controls the electronic zoom. Hereinafter, a toggle operation that toggles from the mode that controls the optical zoom to the mode that controls the electronic zoom, like an operation on the electronic zoom toggle switch 128, is designated the “toggle operation to the mode that controls the electronic zoom” or the “first toggle operation”.

The electronic zoom is a zoom method that changes the zoom magnification by executing image processing on an image, without moving a lens included in the optical system 120a. In the case in which the electronic zoom is executed, a partial region of an image is clipped, and after executing various processes, such as an interpolation process, on the clipped region as appropriate, the image of the region is enlarged. In the case in which the electronic zoom is executed, unlike the case in which the optical zoom is executed, it is not necessary to physically move the zoom lens included in the optical system 120a, and since the zoom magnification is changed by image processing, the zoom magnification is changed discontinuously. Thus, in the case in which the electronic zoom is executed, it is possible to change to the desired zoom magnification in a shorter time than the case in which the optical zoom is executed, and also discontinuously.

In the case in which the medical observation apparatus 100 operates in the mode that controls the optical zoom, the zoom magnification is changed by moving the lens included in the optical system 120a. In the case in which the zoom magnification is changed by the mode that controls the optical zoom, the zoom magnification is not changed by the electronic zoom. Also, in the case in which the medical observation apparatus 100 operates in the mode that controls the electronic zoom, the zoom magnification is changed by image processing. In the case in which the zoom magnification is changed by the mode that controls the electronic zoom, the zoom magnification is not changed by the optical zoom. In other words, in the case of toggling from the mode that controls the optical zoom to the mode that controls the electronic zoom, in the medical observation apparatus 100, as described later, the electronic zoom is executed with respect to a medical captured image at the corresponding optical magnification of the optical system 120a of the imaging device 106 when the toggle operation to the mode that controls the electronic zoom was detected.

The operating mode change switch 130 is an example of an operating device for changing the operating mode of the arm 104 in the imaging device 106. By performing an operation on the operating mode change switch 130, the operating mode of the arm 104 is changed. Examples of operating modes of the arm 104 include a locked mode and a free mode, as described above.

One example of an operation with respect to the operating mode change switch 130 is an operation of pressing the operating mode change switch 130. For example, the operating mode of the arm 104 becomes the free mode while the surgeon is pressing the operating mode change switch 130, and the operating mode of the arm 104 becomes the locked mode when the surgeon is not pressing the operating mode change switch 130.

In addition, the imaging device 106 is provided with, for example, an anti-slip member 132 and a projecting member 134 in order to further raise operability, convenience, and the like when an operator who performs operations on various operation devices performs an operation.

The anti-slip member 132 is a member provided to prevent slipping of an operating body such as a hand when, for example, the operator performs an operation on the barrel member 122 with the operating body. The anti-slip member 132 is formed with a material having a large coefficient of friction, for example, and has a slip-resistant structure due to unevenness or the like.

The projecting member 134 is member provided to prevent an operating body such as a hand blocking the field of view of the optical system 120a when the operator performs an operation on the barrel member 122 with the operating body, or to prevent a cover glass (not illustrated) from becoming dirty due to the cover glass being contacted by the operating body when an operation is performed with the operating body.

Note that the position and shape in which each of the anti-slip member 132 and the projecting member 134 is provided obviously are not limited to the example illustrated in FIG. 4. In addition, the imaging device 106 does not have to be provided with one or both of the anti-slip member 132 and the projecting member 134.

The image signal (image data) generated by imaging in the imaging device 106 is subjected to image processing in a processor that functions as the control section described later, for example. Examples of image processing according to the present embodiment include one or multiple processes from among various processes such as gamma correction, white balance adjustment, image enlargement or reduction related to the electronic zoom function, and pixel interpolation, for example. Note that in the case in which the medical observation system according to the present embodiment includes a control apparatus (not illustrated) that controls various operations in the medical observation apparatus 100, the image processing according to the present embodiment may also be performed in the control apparatus (not illustrated).

For example, the medical observation apparatus 100 transmits a display control signal and the image signal subjected to imaging processing as above to the display apparatus 200.

By transmitting the display control signal and the image signal to the display apparatus 200, on the display screen of the display apparatus 200, a medical captured image in which the observation target is imaged (for example, a taken image in which the operating site is imaged) is displayed enlarged or reduced at a desired magnification by one or both of the optical zoom function and the electronic zoom function.

The medical observation apparatus 100 includes the hardware configuration illustrated with reference to FIGS. 1 and 4, for example.

Note that the hardware configuration of the medical observation apparatus according to the present embodiment is not limited to the configuration illustrated with reference to FIGS. 1 and 4.

For example, the medical observation apparatus according to the present embodiment may also be a configuration not provided with the base 102, in which the arm 104 is directly attached to the ceiling, a wall, or the like of the operating room or the like. For example, in the case in which the arm 104 is attached to the ceiling, the medical observation apparatus according to the present embodiment becomes a configuration in which the arm 104 hangs down from the ceiling.

Also, although FIG. 1 illustrates an example configured so that six degrees of freedom are realized with respect to the driving of the imaging device 106, the configuration of the arm 104 is not limited to a configuration whereby the degrees of freedom with respect to the driving of the imaging device 106 become six degrees of freedom. For example, it is sufficient to configure the arm 104 so that the imaging device 106 can move appropriately in accordance with the application, and factors such as the number and arrangement of joint sections and links, and the directions of the drive shafts of the joint sections can be set appropriately so that the arm 104 has the desired degrees of freedom. To give an example, the medical observation apparatus according to the present embodiment may be a simpler configuration that only allows for X- and Y-axis control, like an ophthalmic microscope or the like.

Also, although FIGS. 1 and 4 illustrate an example in which various types of operating devices for controlling the operation of the imaging device 106 are provided on the imaging device 106, some or all of the operating devices illustrated in FIGS. 1 and 4 may also not be provided on the imaging device 106. To give one example, the various types of operating devices for controlling the operation of the imaging device 106 may also be provided in another part other than the imaging device 106 included in the medical observation apparatus according to the present embodiment. Also, to give another example, the various types of operating devices for controlling the operation of the imaging device 106 may also be external operating devices, such as a footswitch or a remote controller. To give an example of the use case illustrated in FIGS. 2 and 3, the surgeon OP is able to use a footswitch FS (one example of an external operating device) to perform an operation for controlling the action of the imaging device 106, such as a toggle operation to the mode that controls the electronic zoom.

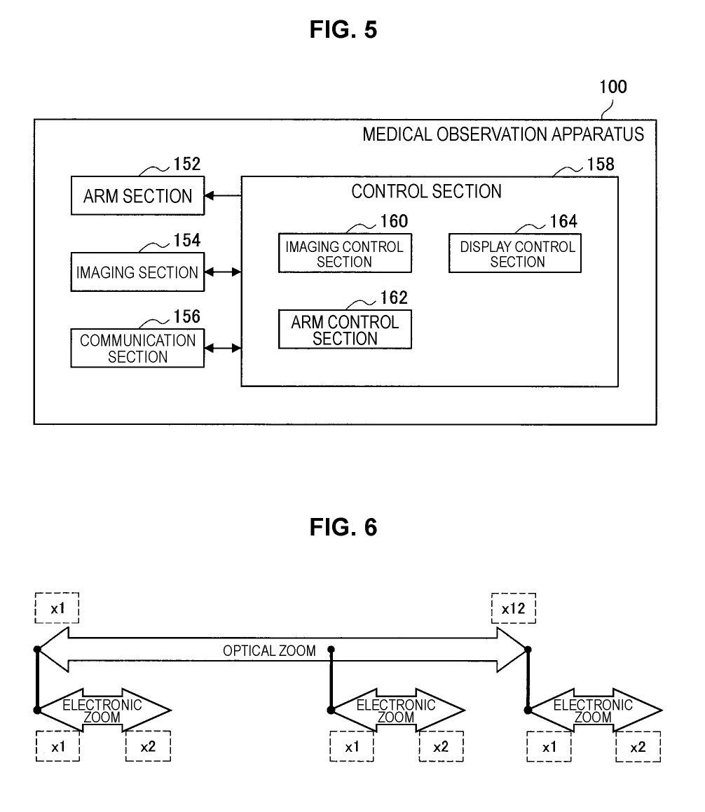

Next, the medical observation apparatus 100 illustrated in FIG. 1 will be described using function blocks. FIG. 5 is a function block diagram illustrating an example of the configuration of the medical observation apparatus 100 according to the present embodiment.

For example, the medical observation apparatus 100 is provided with an arm section 152, an imaging section 154, a communication section 156, and a control section 158.

### [1-2-4] Arm Section 152

The arm section 152 includes the arm 104, and supports the imaging device 106 included in the imaging section 154.

### [1-2-5] Imaging Section 154

The imaging section 154 includes the imaging device 106, and images an observation target. Imaging in the imaging section 154 is controlled by the control section 158, for example.

### [1-2-6] Communication Section 156

The communication section 156 is a communication device provided in the medical observation apparatus 100, and fulfills a role of communicating in a wireless or wired manner with an external apparatus such as the display apparatus 200. The communication section 156 includes the communication device (not illustrated) described above, for example. Communication in the communication section 156 is controlled by the control section 158, for example.

### [1-2-7] Control Section 158

The control section 158 includes the processor (not illustrated) described above, for example, and fulfills a role of controlling the medical observation apparatus 100 overall. In addition, the control section 158 fulfills a role of leading the execution of the processes related to the zoom control method described later.

Note that the processes according to the zoom control method in the control section 158 may also be performed in a distributed manner by multiple processing circuits (such as multiple processors, for example).

More specifically, the control section 158 includes an imaging control section 160, an arm control section 162, and a display control section 164, for example.

### [1-2-7-1] Imaging Control Section 160

The imaging control section 160 controls the imaging device 106 included in the imaging section 154. Examples of the control of the imaging device 106 include control of one or multiple functions typically provided in an electronic imaging microscope section, including at least a zoom function (an optical zoom function and an electronic zoom function), such as control of an AF function.

Also, the imaging control section 160 controls the zoom function of the imaging device 106 by executing the process related to the zoom control method according to the present embodiment.

Hereinafter, an example of the process related to the zoom control method according to the present embodiment will be described.

As described above, in the optical zoom, it is necessary to physically move the zoom lens. For this reason, in the case in which the zoom magnification is changed by optical zoom only, like in an optical medical observation apparatus, for example, it takes approximately several seconds to more than ten seconds until the surgical field desired by the surgeon is obtained.

Accordingly, the imaging control section 160 controls the zoom function of the imaging device 106 so that the electronic zoom is executed at an arbitrary optical magnification of the optical system 120a of the imaging device 106.

FIG. 6 is an explanatory diagram for describing an example of the process related to the zoom control method according to the present embodiment. FIG. 6 illustrates an example in which the optical magnification of the optical system 120a of the imaging device 106 may be changed continuously from a magnification of 1 (“×1” illustrated in FIG. 6) to a magnification of 12 (“×12” illustrated in FIG. 6). Also, FIG. 6 illustrates an example in which the electronic magnification of the electronic zoom of the imaging device 106 may be changed discontinuously to a magnification of 1 (“×1” illustrated in FIG. 6) or a magnification of 2 (“×2” illustrated in FIG. 6). Note that each of the optical magnification of the optical system 120a of the imaging device 106 and the electronic magnification of the imaging device 106 is not limited to the example illustrated in FIG. 6. For example, the optical magnification of the optical system 120a of the imaging device 106 may change depending on the performance of the optical system 120a. Also, as described above, since the electronic zoom is a zoom method that changes the zoom magnification by executing image processing without moving a lens included in the optical system 120a, the electronic magnification of the electronic zoom may change depending on the image processing algorithm or the like, for example.

As illustrated in FIG. 6, in the imaging device 106 provided in the medical observation apparatus 100, the electronic zoom may be executed at an arbitrary optical magnification.

As described above, in the case in which the electronic zoom is executed, it is possible to change to the desired zoom magnification in a shorter time than the case in which the optical zoom is executed, and also discontinuously. Consequently, by having the imaging control section 160 control the zoom function of the imaging device 106 so that the electronic zoom is executed at an arbitrary optical magnification in the imaging device 106, it is possible to shorten the time taken for zooming of a medical captured image.

Also, by potentially shortening the time taken for zooming of a medical captured image, it is possible to decrease the possibility of lowered surgical efficiency like in the case in which an optical medical observation apparatus is used for surgery.

More specifically, as the process related to the zoom control method, the imaging control section 160 executes a process related to a first example indicated in (1) below, and a process related to a second example indicated in (2) below.

### (1) First Example of Process Related to Zoom Control Method

The imaging control section 160 controls the zoom function of the imaging control section 160 on the basis of the toggle operation to the mode that controls the electronic zoom (first toggle operation). The imaging control section 160 controls the zoom function so that the electronic zoom is executed with respect to a predetermined region in the medical captured image captured by the imaging device 106 at the corresponding optical magnification of the optical system 120a of the imaging device 106 when the toggle operation to the mode that controls the electronic zoom was detected.

Herein, the corresponding optical magnification of the optical system 120a of the imaging device 106 when the toggle operation to the mode that controls the electronic zoom was detected may change depending on whether or not the zoom lens included in the optical system 120a is moving when the toggle operation to the mode that controls the electronic zoom was detected, for example. This is because, as described above, with the optical zoom, since it is necessary to physically move the zoom lens, the zoom lens may move in the time between when the control for stopping the moving zoom lens is started until the zoom lens actually stops.

To give an example, in the case in which the zoom lens is not moving when the toggle operation to the mode that controls the electronic zoom is detected, the corresponding optical magnification of the optical system 120a of the imaging device 106 when the toggle operation to the mode that controls the electronic zoom was detected is the optical magnification at the time when the toggle operation to the mode that controls the electronic zoom was detected. Also, in the case in which the zoom lens is moving when the toggle operation to the mode that controls the electronic zoom is detected, the corresponding optical magnification of the optical system 120a of the imaging device 106 when the toggle operation to the mode that controls the electronic zoom was detected is the optical magnification at the time when the movement of the zoom lens stops.

The toggle operation to the mode that controls the electronic zoom may be, for example, an operation on an operating device provided in the medical observation apparatus 100, such as the electronic zoom toggle switch 128. The imaging control section 160 controls the zoom function on the basis of an operation signal output from the operating device provided in the medical observation apparatus 100 in response to the toggle operation to the mode that controls the electronic zoom.

The imaging control section 160 detects the toggle operation to the mode that controls the electronic zoom by detecting a signal pattern corresponding to the toggle operation to the mode that controls the electronic zoom from the operation signal output from the operating device provided in the medical observation apparatus 100, for example. Note that the method of detecting the toggle operation to the mode that controls the electronic zoom in the imaging control section 160 obviously is not limited to the example illustrated above.

Note that the toggle operation to the mode that controls the electronic zoom according to the present embodiment is not limited to the example illustrated above.

For example, the toggle operation to the mode that controls the electronic zoom may also be an operation on an external operating device such as the footswitch FS. The imaging control section 160 detects the toggle operation to the mode that controls the electronic zoom by detecting a signal pattern corresponding to the toggle operation to the mode that controls the electronic zoom from the operation signal output from the external operating device, for example. In the case in which the toggle operation to the mode that controls the electronic zoom is an operation on the footswitch FS, since the surgeon is able to perform the toggle operation to the mode that controls the electronic zoom without taking one's hands away from the surgical field, it is possible to potentially improve the surgical efficiency.

As another example, the toggle operation to the mode that controls the electronic zoom may also be a speech operation performed with respect to a speech input device such as a microphone. The speech input device on which the toggle operation to the mode that controls the electronic zoom is performed may be, for example, one or both of a speech input device provided in the medical observation apparatus 100, and a speech input device external to the medical observation apparatus 100.

The imaging control section 160 executes a speech recognition process on a speech signal output from the speech input device, and controls the zoom function on the basis of a recognition result of the toggle operation to the mode that controls the electronic zoom. In the case in which a command corresponding to the toggle operation to the mode that controls the electronic zoom is recognized from the speech expressed by the speech signal, the imaging control section 160 controls the zoom function. Also, in the case in which a command corresponding to the toggle operation to the mode that controls the electronic zoom is not recognized from the speech expressed by the speech signal, the imaging control section 160 does not control the zoom function. In the case in which the toggle operation to the mode that controls the electronic zoom is a speech operation, since the surgeon is able to perform the toggle operation to the mode that controls the electronic zoom without taking one's hands away from the surgical field, it is possible to potentially improve the surgical efficiency.

For example, the imaging control section 160 controls the zoom function of the imaging device 106 on the basis of one or multiple operations from among the toggle operation to the mode that controls the electronic zoom with respect to an operating device provided in the medical observation apparatus 100, the toggle operation to the mode that controls the electronic zoom with respect to an external operating device, and the toggle operation to the mode that controls the electronic zoom by speech input into a speech input device. Note that the toggle operation to the mode that controls the electronic zoom according to the present embodiment may also be an arbitrary operation by which the medical observation apparatus 100 is capable of detecting that the toggle operation to the mode that controls the electronic zoom has been performed, such as a gesture.

Examples of the predetermined region in the medical captured image on which the imaging control section 160 executes the electronic zoom may be, for example, the region related to the first example indicated in (A) below to the region related to the third example indicated in (C) below.

(A) First Example of Predetermined Region: Preset Region with Respect to Medical Captured Image

The predetermined region may be a preset region with respect to the medical captured image. The imaging control section 160 controls the zoom function so that the electronic zoom is executed on the preset region with respect to the medical captured image.

For example, the imaging control section 160 specifies the predetermined region by referencing region information indicating the preset region (for example, data indicating the shape of the region, the center position of the region, and the size of the region) which is stored in a recording medium such as the storage section (not illustrated). Subsequently, the imaging control section 160 executes the electronic zoom on the specified predetermined region.

The preset region may be, for example, a region centered on the center position of the medical captured image. The region centered on the center position of the medical captured image may be, for example, a rectangular region centered on the center position of the medical captured image. Also, the size of the region centered on the center position of the medical captured image may be a preset, fixed size, or may be a size which is changeable by an operation from a user (for example, a medical personnel member such as a surgeon or a surgeon's assistant) who uses the medical observation apparatus 100.

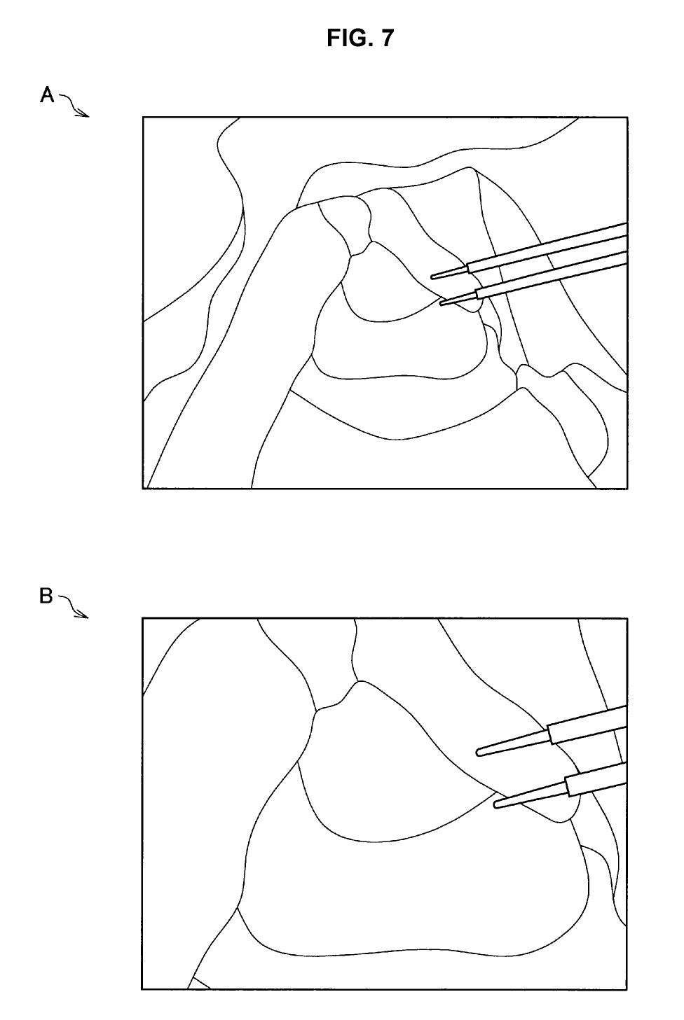

Note that the preset region is not limited to the example given above, and may also be a region centered on an arbitrary position in the medical captured image. FIG. 7 is an explanatory diagram for describing an example of the process related to the zoom control method according to the present embodiment. FIG. 7 illustrates an example of a medical captured image displayed on the display screen of the display apparatus 200 in the case in which the electronic zoom is executed on the preset region with respect to the medical captured image. A of FIG. 7 illustrates an example of a medical captured image displayed on the display screen of the display apparatus 200 before the toggle operation to the mode that controls the electronic zoom is performed, or in other words, illustrates an example of a medical captured image on which the electronic zoom has not been executed. Also, B of FIG. 7 illustrates an example of a medical captured image displayed on the display screen of the display apparatus 200 after the toggle operation to the mode that controls the electronic zoom is performed, or in other words, illustrates an example of a medical captured image on which the electronic zoom has been executed.

If the toggle operation to the mode that controls the electronic zoom is detected, the imaging control section 160 executes the electronic zoom on the preset region. In the case in which the preset region is “a region centered on the center position of the medical captured image”, the medical captured image displayed on the display screen of the display apparatus 200 as a result of the electronic zoom being executed changes from the medical captured image illustrated in A of FIG. 7 to the medical captured image illustrated in B of FIG. 7.

As described above, by executing the electronic zoom, the zoom magnification is changed to the desired zoom magnification in a shorter time than the case in which the optical zoom is executed, and also discontinuously. Thus, after the toggle operation to the mode that controls the electronic zoom is performed, the time taken to change from the medical captured image illustrated in A of FIG. 7 to the medical captured image illustrated in B of FIG. 7 becomes a shorter time than in the case of executing the optical zoom.

### (B) Second Example of Predetermined Region: Region Set on the Basis of Detection Result of Gaze Detection Sensor 300

The predetermined region may be a region set on the basis of a detection result from the gaze detection sensor 300. The imaging control section 160 sets the predetermined region on the basis of information indicating a gaze detection result output from the gaze detection sensor 300 when the toggle operation to the mode that controls the electronic zoom is detected. The information indicating the gaze detection result from when the toggle operation to the mode that controls the electronic zoom is detected may be, for example, information indicating the gaze detection result acquired first after the toggle operation to the mode that controls the electronic zoom is detected, or information indicating the gaze detection result acquired most recently with reference to the time at which the toggle operation to the mode that controls the electronic zoom is detected.

For example, the imaging control section 160 specifies a gaze position on the display screen (the position of the gaze of the detection target of the gaze detection sensor 300) on the basis of the information indicating the gaze detection result from when the toggle operation to the mode that controls the electronic zoom is detected. When the gaze position on the display screen is specified, the imaging control section 160 sets the predetermined region on the basis of the specified gaze position on the display screen. Subsequently, the imaging control section 160 executes the electronic zoom on the set predetermined region.

For example, the imaging control section 160 converts the specified gaze position on the display screen to a gaze position on the medical captured image, and sets a region centered on the gaze position in the medical captured image as the predetermined region. The imaging control section 160 converts the gaze position on the display screen to a gaze position on the medical captured image by an arbitrary method enabling conversion of the gaze position on the display screen to the gaze position on the medical captured image, such as “executing the computations of an arbitrary algorithm that converts a gaze position on the display screen to a gaze position on the medical captured image”, “referencing a table (or database) associating positions on the display screen with positions on the medical captured image”, or the like.

The region centered on the gaze position in the medical captured image may be, for example, a rectangular region centered on the gaze position. Also, the region centered on the gaze position may be, for example, “a region which does not exceed a preset size, but is a maximum size which can be set with respect to the medical captured image”.

If the toggle operation to the mode that controls the electronic zoom is detected, the imaging control section 160 sets the predetermined region on the basis of the information indicating the gaze detection result from when the toggle operation to the mode that controls the electronic zoom was detected, and executes the electronic zoom on the set region. By executing the electronic zoom on the set region, the medical captured image displayed on the display screen of the display apparatus 200 changes to the medical captured image after the electronic zoom is executed.

As described above, by executing the electronic zoom, the zoom magnification is changed to the desired zoom magnification in a shorter time than the case in which the optical zoom is executed, and also discontinuously. Thus, after the toggle operation to the mode that controls the electronic zoom is performed, the time taken to change to the medical captured image after the electronic zoom is executed becomes a shorter time than in the case of executing the optical zoom.

### (C) Third Example of Predetermined Region: Region Set on the Basis of Medical Captured Image

The predetermined region may be a region set on the basis of the medical captured image. The imaging control section 160 sets the predetermined region on the basis of the medical captured image from when the toggle operation to the mode that controls the electronic zoom is detected. The medical captured image from when the toggle operation to the mode that controls the electronic zoom is detected may be, for example, “the medical captured image that was displayed on the display screen of the display apparatus 200 at the time when the toggle operation to the mode that controls the electronic zoom was detected”, or “the medical captured image obtained from the imaging device 106 after the toggle operation to the mode that controls the electronic zoom was detected”.

More specifically, the imaging control section 160 detects a treatment tool from the medical captured image from when the toggle operation to the mode that controls the electronic zoom is detected, and sets the predetermined region on the basis of a position corresponding to the treatment tool in the medical captured image. Subsequently, the imaging control section 160 executes the electronic zoom on the set predetermined region.

For example, the imaging control section 160 detects the treatment tool from the medical captured image by referencing data indicating features of the treatment tool (such as data indicating the shape of the treatment tool, for example) which is stored in a recording medium such as the storage section (not illustrated), and executing a process related to pattern recognition. Note that the method of detecting the treatment tool from the medical captured image is not limited to the example given above, and the imaging control section 160 may also use an arbitrary method enabling detection of the treatment tool from the medical captured image.

The position corresponding to the treatment tool in the medical captured image may be, for example, the position of an affected area relative to the position of the treatment tool in the medical captured image. The reason why the position of the affected area relative to the position of the treatment tool in the medical captured image is treated as the position corresponding to the treatment tool in the medical captured image is because the region of the medical captured image that the surgeon wants to focus on is thought to be the affected area, and not the treatment tool itself.

Note that the position corresponding to the treatment tool in the medical captured image may also be the position of the treatment tool detected from the medical captured image.

In the following, an example will be given for a case in which the imaging control section 160 treats the position of the affected area relative to the position of the treatment tool in the medical captured image as the position corresponding to the treatment tool in the medical captured image. Also, in the following, the position of the affected area relative to the position of the treatment tool in the medical captured image will be designated the “relative position of the affected area” in some cases.

The imaging control section 160 estimates the relative position of the affected area on the basis of the medical captured image, and treats the estimated relative position of the affected area as the position corresponding to the treatment tool. When the position corresponding to the treatment tool is specified, the imaging control section 160 sets a region centered on the position corresponding to the treatment tool as the predetermined region, for example. Subsequently, the imaging control section 160 executes the electronic zoom on the set predetermined region.

The region centered on the position corresponding to the treatment tool may be, for example, a rectangular region centered on the position corresponding to the treatment tool. Also, the region centered on the position corresponding to the treatment tool may be, for example, “a region which does not exceed a preset size, but is a maximum size which can be set with respect to the medical captured image”.

(C-1) Example of Process for Case in which Treatment Tool Detected from Medical Captured Image is a Pair of Retractors

In the case in which the treatment tool detected from the medical captured image is a pair of retractors, the imaging control section 160 estimates an intermediate position between the pair of retractors as the relative position of the affected area. The reason for estimating an intermediate position between the pair of retractors as the relative position of the affected area is because, in the case in which the retractors are used for surgery, the affected area, such as a tumor or an aneurysm, generally exists between the pair of retractors.

Additionally, the imaging control section 160 executes the electronic zoom on the region centered on the intermediate position between the pair of retractors (one example of the position corresponding to the treatment tool).

Herein, the intermediate position between the pair of retractors may be, for example, “the midpoint position of a line segment joining an arbitrary point on each of the pair of retractors detected from the medical captured image”. In addition, the intermediate position between the pair of retractors may also be a position between the pair of retractors specified by an arbitrary method, such as “the center position (or center-of-gravity position) of a region prescribed by two arbitrary points on each of the pair of retractors (for a total of four points) detected from the medical captured image”.

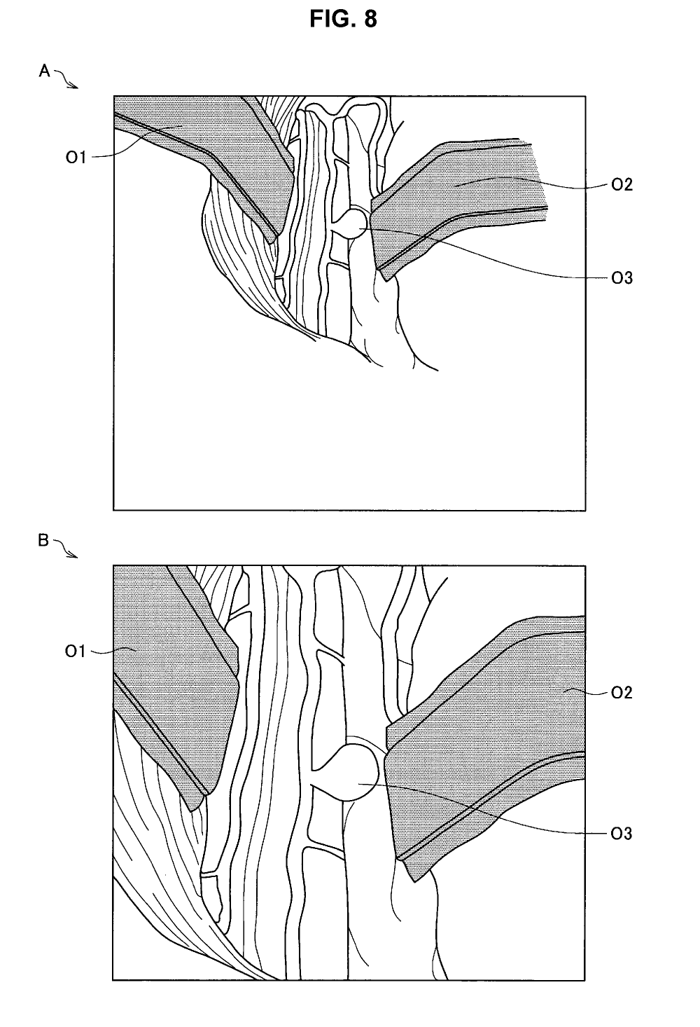

FIG. 8 is an explanatory diagram for describing another example of a process related to a zoom control method according to the present embodiment. FIG. 8 illustrates an example of a medical captured image displayed on the display screen of the display apparatus 200 in the case in which the electronic zoom is executed on a region centered on the intermediate position between the pair of retractors. A of FIG. 8 illustrates an example of a medical captured image displayed on the display screen of the display apparatus 200 before the toggle operation to the mode that controls the electronic zoom is performed, or in other words, illustrates an example of a medical captured image on which the electronic zoom has not been executed. Also, B of FIG. 8 illustrates an example of a medical captured image displayed on the display screen of the display apparatus 200 after the toggle operation to the mode that controls the electronic zoom is performed, or in other words, illustrates an example of a medical captured image on which the electronic zoom has been executed.

O1 and O2 illustrated in FIG. 8 indicate the pair of retractors. Also, O3 illustrated in FIG. 8 indicates the affected area.

If the toggle operation to the mode that controls the electronic zoom is detected, the imaging control section 160 detects the pair of retractors from the medical captured image from when the toggle operation to the mode that controls the electronic zoom was detected. If the pair of retractors is detected, the imaging control section 160 estimates the intermediate position between the pair of retractors as the position of the affected area. Subsequently, the imaging control section 160 executes the electronic zoom on a region centered on the intermediate position between the pair of retractors, for example.

As a result of the electronic zoom being executed on the region centered on the intermediate position between the pair of retractors, the medical captured image displayed on the display screen of the display apparatus 200 changes from the medical captured image illustrated in A of FIG. 8 to the medical captured image illustrated in B of FIG. 8.

As described above, by executing the electronic zoom, the zoom magnification is changed to the desired zoom magnification in a shorter time than the case in which the optical zoom is executed, and also discontinuously. Thus, after the toggle operation to the mode that controls the electronic zoom is performed, the time taken to change from the medical captured image illustrated in A of FIG. 8 to the medical captured image illustrated in B of FIG. 8 becomes a shorter time than in the case of executing the optical zoom.

Also, as illustrated in A of FIG. 8, in the case in which retractors are used for surgery, the affected area O3 often exists between the pair of retractors O1 and O2. As a result of the electronic zoom being executed on the region centered on the intermediate position between the pair of retractors, as illustrated in B of FIG. 8, in the medical captured image after the electronic zoom is executed, the affected area O3 is positioned near the center of the medical captured image. Thus, by executing the electronic zoom on the region centered on the intermediate position between the pair of retractors, the enlargement by electronic zoom of the region of the medical captured image that the surgeon wants to focus on is realized.

(C-2) Example of Process for Case in which Treatment Tool Detected from Medical Captured Image is an Energy Treatment Tool

In the case in which the treatment tool detected from the medical captured image is an energy treatment tool, such as an electrosurgical instrument, a bipolar instrument, or an ultrasonic aspirator, the imaging control section 160 estimates the front edge position of the energy treatment tool as the position of the affected area. The front edge position of an energy treatment tool is, for example, the position of the front edge of the portion of the energy treatment tool used to perform a treatment on an affected area. The reason for estimating the front edge position of the energy treatment tool as the relative position of the affected area is because, in the case in which the energy treatment tool is used for surgery, the affected area often exists near the front edge position of the energy treatment tool.

If the toggle operation to the mode that controls the electronic zoom is detected, the imaging control section 160 detects the energy treatment tool from the medical captured image from when the toggle operation to the mode that controls the electronic zoom was detected. If the energy treatment tool is detected, the imaging control section 160 estimates the front edge position of the energy treatment tool as the position of the affected area. Additionally, the imaging control section 160 executes the electronic zoom on the region centered on the front edge position of the energy treatment tool (one example of the position corresponding to the treatment tool), for example.

By executing the electronic zoom on the region centered on the front edge position of the energy treatment tool, the medical captured image displayed on the display screen of the display apparatus 200 changes to the medical captured image after the electronic zoom is executed.

As described above, by executing the electronic zoom, the zoom magnification is changed to the desired zoom magnification in a shorter time than the case in which the optical zoom is executed, and also discontinuously. Thus, after the toggle operation to the mode that controls the electronic zoom is performed, the time taken to change to the medical captured image after the electronic zoom is executed becomes a shorter time than in the case of executing the optical zoom.

Also, in the case in which the energy treatment tool is used for surgery, the affected area often exists near the front edge position of the energy treatment tool. For this reason, as a result of the electronic zoom being executed on the region centered on the front edge position of the energy treatment tool, in the medical captured image after the electronic zoom is executed, there is a high probability that the affected area is positioned near the center of the medical captured image. Thus, by executing the electronic zoom on the region centered on the front edge position of the energy treatment tool, the enlargement by electronic zoom of the region of the medical captured image that the surgeon wants to focus on may be realized.

(C-3) Example of Process for Case in which Treatment Tool Detected from Medical Captured Image are Forceps

In the case in which the treatment tool detected from the medical captured image are forceps, the imaging control section 160 estimates the front edge position of the forceps as the position of the affected area. The front edge position of the forceps is, for example, the position of the front edge of the portion of the forceps used to perform a treatment on the affected area. The reason for estimating the front edge position of the forceps as the relative position of the affected area is because, in the case in which the forceps are used for surgery, the affected area often exists near the front edge position of the forceps.

If the toggle operation to the mode that controls the electronic zoom is detected, the imaging control section 160 detects the forceps from the medical captured image from when the toggle operation to the mode that controls the electronic zoom was detected. If the forceps are detected, the imaging control section 160 estimates the front edge position of the forceps as the position of the affected area. Additionally, the imaging control section 160 executes the electronic zoom on the region centered on the front edge position of the forceps (one example of the position corresponding to the treatment tool), for example.

By executing the electronic zoom on the region centered on the front edge position of the forceps, the medical captured image displayed on the display screen of the display apparatus 200 changes to the medical captured image after the electronic zoom is executed.

As described above, by executing the electronic zoom, the zoom magnification is changed to the desired zoom magnification in a shorter time than the case in which the optical zoom is executed, and also discontinuously. Thus, after the toggle operation to the mode that controls the electronic zoom is performed, the time taken to change to the medical captured image after the electronic zoom is executed becomes a shorter time than in the case of executing the optical zoom.

Also, in the case in which the forceps are used for surgery, the affected area often exists near the front edge position of the forceps. For this reason, as a result of the electronic zoom being executed on the region centered on the front edge position of the forceps, in the medical captured image after the electronic zoom is executed, there is a high probability that the affected area is positioned near the center of the medical captured image. Thus, by executing the electronic zoom on the region centered on the front edge position of the forceps, the enlargement by electronic zoom of the region of the medical captured image that the surgeon wants to focus on may be realized.

As indicated in (C-1) to (C-3) above, for example, the imaging control section 160 controls the zoom function so that the electronic zoom is executed on a predetermined region set by detecting a treatment tool from the medical captured image.

Note that the treatment tool targeted by the zoom control method according to the present embodiment is not limited to a pair of retractors, an energy treatment tool, and forceps. For example, the treatment tool targeted by the zoom control method according to the present embodiment may also be an arbitrary treatment tool used in surgery utilizing the medical observation apparatus 100, such as a position detection probe used by a navigation apparatus or an innervation probe used by a nerve monitor.

For example, the imaging control section 160 controls the zoom function so that the electronic zoom is executed on any of the regions from the region related to the first example illustrated in (A) above to the region related to the third example illustrated in (C) above.

For example, the imaging control section 160 references region setting information prescribing a region specification method stored in a recording medium such as the storage section (not illustrated), and by following the region specification method indicated by the region setting information, controls the zoom function so that the electronic zoom is executed on any of the regions from the region related to the first example illustrated in (A) above to the region related to the third example illustrated in (C) above. For example, by changing the region specification method indicated by the region setting information, the imaging control section 160 is able to change the method of specifying a region.

Also, the imaging control section 160 may control the zoom function so that the electronic zoom is executed on a region of high priority among the regions from the region related to the first example illustrated in (A) above to the region related to the third example illustrated in (C) above. To give an example, in the case in which the region related to the third example illustrated in (C) above is of higher priority than the region related to the first example illustrated in (A) above, the imaging control section 160 first executes the process indicated in (C) above, and sets the predetermined region on the basis of the medical captured image. In the case in which the predetermined region is set successfully on the basis of the medical captured image, the electronic zoom is executed on the set predetermined region. Also, in the case in which the predetermined region is not set successfully on the basis of the medical captured image, the process indicated in (A) above is executed, and the electronic zoom is executed on the preset region.

### (2) Second Example of Process Related to Zoom Control Method

The imaging control section 160 cancels the electronic zoom on the basis of a toggle operation that toggles from the mode that controls the electronic zoom to the mode that controls the optical zoom. Canceling the electronic zoom according to the present embodiment refers to, for example, reverting the zoom function of the imaging device 106 back to the state before the control based on the toggle operation to the mode that controls the electronic zoom indicated in (1) above is executed. In the following, a toggle operation that toggles from the mode that controls the electronic zoom to the mode that controls the optical zoom is designated the “toggle operation to the mode that controls the optical zoom” or the “second toggle operation”.

The toggle operation to the mode that controls the optical zoom may be, for example, an operation on an operating device related to the optical zoom provided in the medical observation apparatus 100, such as the zoom switch 124. The operating device related to the optical zoom may also be a dedicated switch for toggling from the mode that controls the electronic zoom to the mode that controls the optical zoom (hereinafter designated the “optical zoom toggle switch” in some cases). The imaging control section 160 cancels the electronic zoom on the basis of an operation signal output from the operating device provided in the medical observation apparatus 100 in response to the toggle operation to the mode that controls the optical zoom.

The imaging control section 160 detects the toggle operation to the mode that controls the optical zoom by detecting a signal pattern corresponding to the toggle operation to the mode that controls the optical zoom from the operation signal output from the operating device provided in the medical observation apparatus 100, for example. Note that the method of detecting the toggle operation to the mode that controls the optical zoom in the imaging control section 160 obviously is not limited to the example illustrated above.

Note that the toggle operation to the mode that controls the optical zoom according to the present embodiment is not limited to the example illustrated above.

For example, the toggle operation to the mode that controls the optical zoom may also be an operation on an external operating device such as the footswitch FS. The imaging control section 160 detects the toggle operation to the mode that controls the optical zoom by detecting a signal pattern corresponding to the toggle operation to the mode that controls the optical zoom from the operation signal output from the external operating device, for example. In the case in which the toggle operation to the mode that controls the optical zoom is an operation on the footswitch FS, since the surgeon is able to perform the toggle operation to the mode that controls the optical zoom without taking one's hands away from the surgical field, it is possible to potentially improve the surgical efficiency.

As another example, the toggle operation to the mode that controls the optical zoom may also be a speech operation performed with respect to a speech input device. The speech input device on which the toggle operation to the mode that controls the optical zoom is performed may be, for example, one or both of a speech input device provided in the medical observation apparatus 100, and a speech input device external to the medical observation apparatus 100.

The imaging control section 160 executes a speech recognition process on a speech signal output from the speech input device, and cancels the electronic zoom on the basis of a recognition result of the toggle operation to the mode that controls the optical zoom. In the case in which a command corresponding to the toggle operation to the mode that controls the optical zoom is recognized from the speech expressed by the speech signal, the imaging control section 160 cancels the electronic zoom. Also, in the case in which a command corresponding to the toggle operation to the mode that controls the optical zoom is not recognized from the speech expressed by the speech signal, the imaging control section 160 does not cancel the electronic zoom. In the case in which the toggle operation to the mode that controls the optical zoom is a speech operation, since the surgeon is able to perform the toggle operation to the mode that controls the optical zoom without taking one's hands away from the surgical field, it is possible to potentially improve the surgical efficiency.

For example, the imaging control section 160 cancels the electronic zoom of the imaging device 106 on the basis of one or multiple operations from among the toggle operation to the mode that controls the optical zoom with respect to an operating device provided in the medical observation apparatus 100, the toggle operation to the mode that controls the optical zoom with respect to an external operating device, and the toggle operation to the mode that controls the optical zoom by speech input into a speech input device. Note that the toggle operation to the mode that controls the optical zoom according to the present embodiment may also be an arbitrary operation by which the medical observation apparatus 100 is capable of detecting that the toggle operation to the mode that controls the optical zoom has been performed, such as a gesture.

As the process related to the zoom control method, the imaging control section 160 executes the process related to the first example indicated in (1) above, and the process related to the second example indicated in (2) above.

### [1-2-7-2] Arm Control Section 162

The arm control section 162 controls the driving of the arm 104 included in the arm section 152. One example of control of the driving of the arm 104 includes, for example, “applying a control signal that controls driving to the actuators (not illustrated) corresponding to each of the joint sections 110a, 110b, 110c, 110d, 110e, and 110f”, and the like.

### [1-2-7-3] Display Control Section 164

For example, the display control section 164 controls the display on the display apparatus 200 by conveying the display control signal and the image signal to the communication device (not illustrated) included in the communication section 156, and causing the display control signal and the image signal to be transmitted to the display apparatus 200. Note that the control of communication in the communication section 156 may also be performed by a communication control section (not illustrated) included in the control section 158.

For example, by including the imaging control section 160, the control section 158 fulfills a role of leading the execution of the processes related to the zoom control method according to the present embodiment. Also, for example, by including the imaging control section 160, the arm control section 162, and the display control section 164, the control section 158 fulfills a role of controlling the medical observation apparatus 100 overall.

Note that the configuration of the control section 158 is not limited to the example illustrated in FIG. 5.

For example, the control section 158 can include an arbitrary configuration corresponding to how the functions included in the medical observation apparatus 100 are divided up, such as a configuration corresponding to how the processes related to the zoom control method according to the present embodiment are divided up. To give an example, the control section 158 may include, separately from the imaging control section 160, one or both of an operation detection section (not illustrated) that detects each of the toggle operation to the mode that controls the electronic zoom and the toggle operation to the mode that controls the optical zoom, and a region setting section (not illustrated) that sets the predetermined region.

The medical observation apparatus 100 performs processes related to the zoom control method according to the present embodiment described later with the configuration illustrated in FIG. 5, for example.

Note that the configuration of the medical observation apparatus according to the present embodiment is not limited to the configuration illustrated in FIG. 5.

For example, in the medical observation apparatus according to the present embodiment, one or more of the imaging control section 160, the arm control section 162, and the display control section 164 illustrated in FIG. 5 can be provided separately from the control section 158 (for example, realized by a different processing circuit).

Additionally, in the medical observation apparatus according to the present embodiment, the configuration for realizing processes related to the zoom control method according to the present embodiment is not limited to the configuration illustrated in FIG. 5. For example, the medical observation apparatus according to the present embodiment can take a configuration corresponding to how the processes related to the zoom control method according to the present embodiment are divided up.

Also, for example, in the case of communicating with an external apparatus via an external communication device having a function and configuration similar to the communication section 156, the medical observation apparatus according to the present embodiment may also not be provided with the communication section 156.

Also, in the case in which the medical observation system according to the present embodiment includes the control apparatus (not illustrated), and the medical observation apparatus according to the present embodiment is controlled by the control apparatus (not illustrated), the medical observation apparatus according to the present embodiment may also not be provided with the control section 158.

Herein, the control apparatus (not illustrated) is, for example, provided with a control section having a function and configuration similar to the control section 158, and thereby performs processes related to the zoom control method according to the present embodiment, and in addition, controls the operation in each structural element such as the arm section 152 and the imaging section 154 provided in the medical observation apparatus according to the present embodiment. The control apparatus (not illustrated) communicates with the medical observation apparatus according to the present embodiment via a provided communication device or a connected external communication device, and thereby controls the operation in each structural element provided in the medical observation apparatus according to the present embodiment.

Furthermore, in the case in which the medical observation system according to the present embodiment includes the control apparatus (not illustrated), and the medical observation apparatus according to the present embodiment is controlled by the control apparatus (not illustrated), it is also possible for the medical observation apparatus according to the present embodiment to take a configuration that does not include some of the functions of the control section 158.

### [2] Example of Process Related to the Zoom Control Method According to Present Embodiment

Next, an example of processes related to the zoom control method according to the present embodiment will be described. The following gives an example of a case in which the processes related to the zoom control method according to the present embodiment are performed by the medical observation apparatus 100 (more specifically, the imaging control section 160 included in the medical observation apparatus 100, for example). Note that, as described above, in the medical observation system according to the present embodiment, the processes related to the zoom control method according to the present embodiment may also be performed by the control apparatus (not illustrated).

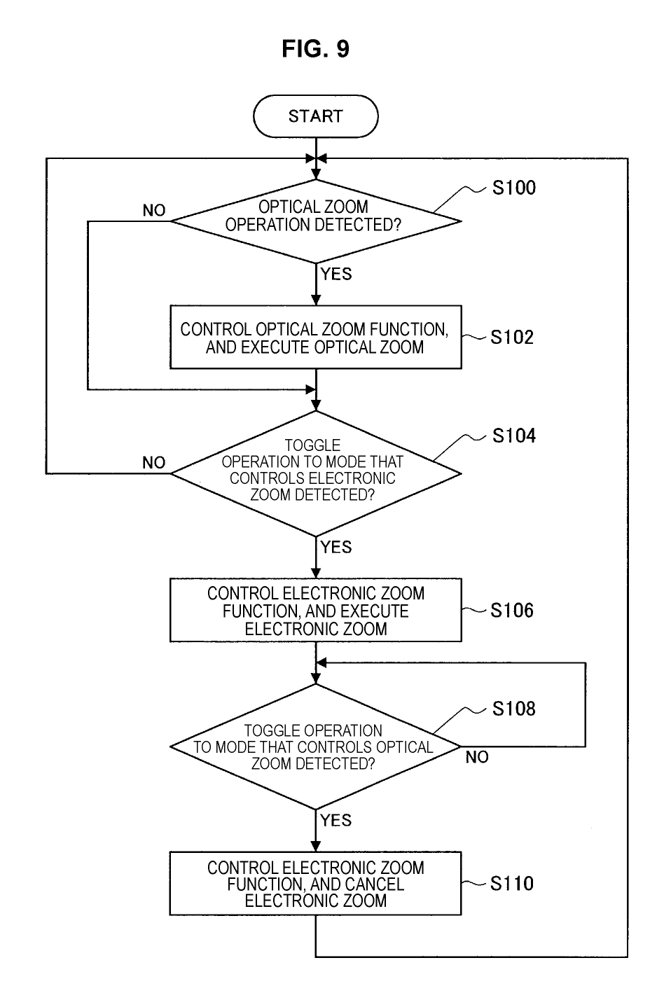

FIG. 9 is a flowchart illustrating an example of the process related to the zoom control method according to the present embodiment. For example, the processes in steps S104 and S106 illustrated in FIG. 9 correspond to processes related to the zoom control method according to the first example indicated in (1) above, while the processes in steps S108 and S110 illustrated in FIG. 9 correspond to processes related to the zoom control method according to the second example indicated in (2) above.

The medical observation apparatus 100 determines whether or not an optical zoom operation has been detected (S100). For example, the medical observation apparatus 100 determines that an optical zoom operation has been detected in the case in which a signal pattern corresponding to an optical zoom operation is detected from an operation signal output from the zoom switch 124 or an operation signal output from an external operating device, such as the footswitch FS.

In step S100, in the case of determining that the optical zoom operation has been detected, the medical observation apparatus 100 executes the process from step S104 described later.

Also, in step S100, in the case of determining that the optical zoom operation has been detected, the medical observation apparatus 100 controls the optical zoom function of the imaging device 106 in response to the detected optical zoom operation, and executes the optical zoom corresponding to the detected optical zoom operation (S102).

In step S100, in the case of not determining that the optical zoom operation has been detected, or in the case in which the process of step S102 has been executed, the medical observation apparatus 100 determines whether or not the toggle operation to the mode that controls the electronic zoom has been detected (S104). For example, the medical observation apparatus 100 determines that the toggle operation to the mode that controls the electronic zoom has been detected in the case in which a signal pattern corresponding to the toggle operation to the mode that controls the electronic zoom is detected from an operation signal output from the electronic zoom toggle switch 128 or an operation signal output from an external operating device, such as the footswitch FS. Additionally, the medical observation apparatus 100 may also determine that the toggle operation to the mode that controls the electronic zoom has been detected in the case in which a command corresponding to the toggle operation to the mode that controls the electronic zoom is recognized from a speech signal output from a speech input device, for example.

In step S104, in the case of not determining that the toggle operation to the mode that controls the electronic zoom has been detected, the medical observation apparatus 100 repeats the process from step S100, for example.

Also, in step S104, in the case of determining that the toggle operation to the mode that controls the electronic zoom has been detected, the medical observation apparatus 100 controls the electronic zoom function of the imaging device 106 in response to the detected toggle operation to the mode that controls the electronic zoom, and executes the electronic zoom corresponding to the detected toggle operation to the mode that controls the electronic zoom (S106). For example, the medical observation apparatus 100 executes the electronic zoom on any of the regions from the region related to the first example illustrated in (A) above to the region related to the third example illustrated in (C) above.

If the process in step S106 is executed, the medical observation apparatus 100 determines whether or not the toggle operation to the mode that controls the optical zoom has been detected (S108). For example, the medical observation apparatus 100 determines that the toggle operation to the mode that controls the optical zoom has been detected in the case in which a signal pattern corresponding to the toggle operation to the mode that controls the optical zoom is detected from an operation signal output from the optical zoom toggle switch or an operation signal output from an external operating device, such as the footswitch FS. Additionally, the medical observation apparatus 100 may also determine that the toggle operation to the mode that controls the optical zoom has been detected in the case in which a command corresponding to the toggle operation to the mode that controls the optical zoom is recognized from a speech signal output from a speech input device, for example.

In step S108, in the case of not determining that the toggle operation to the mode that controls the optical zoom has been detected, the medical observation apparatus 100 does not advance the process until it is determined that the toggle operation to the mode that controls the optical zoom has been detected, for example.

Also, in step S108, in the case of determining that the toggle operation to the mode that controls the optical zoom has been detected, the medical observation apparatus 100 controls the electronic zoom function of the imaging device 106 in response to the detected toggle operation to the mode that controls the optical zoom, and cancels the electronic zoom that was executed in step S106 (S110). Subsequently, the medical observation apparatus 100 repeats the process from step S100.

By executing the process illustrated in FIG. 9, for example, the medical observation apparatus 100 controls the zoom function (optical zoom function and electronic zoom function) of the imaging device 106. Note that the example of the process related to the zoom control method according to the present embodiment obviously is not limited to the example illustrated in FIG. 9.

### [3] Example of Advantageous Effects Exhibited by Use of Medical Information Processing System According to Present Embodiment

By using the medical information processing system according to the present embodiment, the advantageous effects indicated below are exhibited, for example. Note that the advantageous effects exhibited by using the medical information processing system according to the present embodiment obviously are not limited to the examples indicated below.
The medical observation apparatus 100, by using the electronic zoom in conjunction with the optical zoom, is able to present an enlarged view instantaneously. Thus, by using the medical observation apparatus 100, lowered surgical efficiency is prevented.
In the case of toggling from the mode that controls the optical zoom to the mode that controls the electronic zoom, the focus is unaffected, thereby making operations for fine adjustment of the focus unnecessary. Thus, by using the medical observation apparatus 100, there is a possibility of potentially improving the surgical efficiency.
Since an enlarged view is presented instantaneously, and since operations for fine adjustment of the focus are unnecessary, when the surgeon performs the toggle operation to the mode that controls the electronic zoom, an enlarged view is realized without having the surgeon perform optical zoom operations and operations for fine adjustment of the focus. Thus, for example, in the case in which the surgeon performs the toggle operation to the mode that controls the electronic zoom when ligaturing while puncturing with a needle in an angiostomy, there is a possibility that the surgeon is able to perform inosculation efficiently.
In the case in which the medical observation apparatus 100 includes a speech recognition function, since the surgeon is able to operate the zoom function included in the medical observation apparatus 100 by speech, there is a possibility of potentially improving the surgical efficiency.

### (Program According to Present Embodiment)

By having a program (a program capable of executing the process related to the zoom control method according to the present embodiment) for causing a computer system to function as the medical observation apparatus according to the present embodiment (or the control apparatus according to the present embodiment) be executed by a processor or the like in the computer system, it is possible to potentially shorten the time taken for zooming of a medical captured image. Herein, a computer system according to the present embodiment includes a single computer, or multiple computers. A series of processes related to the zoom control method according to the present embodiment is executed by the computer system according to the present embodiment.

Additionally, by having the program for causing a computer system to function as the medical observation apparatus according to the present embodiment (or the control apparatus according to the present embodiment) be executed by a processor or the like in the computer system, the effects exhibited by the display realized by the process related to the zoom control method according to the present embodiment described above can be exhibited.

It should be understood by those skilled in the art that various modifications, combinations, sub-combinations and alterations may occur depending on design requirements and other factors insofar as they are within the scope of the appended claims or the equivalents thereof.

For example, although the above indicates that a program (computer program) for causing a computer system to function as the medical observation apparatus according to the present embodiment is provided, in the present embodiment, the above program may also be provided in conjunction with a recording medium on which the above program is stored.

The configuration described above illustrates one example of the present embodiment, and rightfully belongs to the technical scope of the present disclosure.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.

(1) A medical observation apparatus including:

an imaging control section that controls a zoom function of an imaging device on a basis of a first toggle operation that toggles from a mode that controls an optical zoom to a mode that controls an electronic zoom, in which

the imaging control section controls the zoom function so that the electronic zoom is executed on a predetermined region in a medical captured image captured by the imaging device, at an optical magnification of an optical system of the imaging device corresponding to when the first toggle operation was detected.

(2) The medical observation apparatus according to (1), in which

the predetermined region is a region set in advance with respect to the medical captured image.

(3) The medical observation apparatus according to (2), in which the region set in advance is a region centered on a center position of the medical captured image.
(4) The medical observation apparatus according to (1), in which

the medical captured image is displayed on a display screen, and

the imaging control section sets the predetermined region on a basis of a gaze position of a detection target of a gaze detection sensor on the display screen from when the first toggle operation was detected.

(5) The medical observation apparatus according to (1), in which

the imaging control section

detects a treatment tool from the medical captured image from when the first toggle operation was detected, and

sets the predetermined region on a basis of a position corresponding to the treatment tool in the medical captured image.

(6) The medical observation apparatus according to (5), in which

the imaging control section

estimates a position of an affected area relative to the position of the treatment tool in the medical captured image, and

treats the estimated position of the affected area as the position corresponding to the treatment tool.

(7) The medical observation apparatus according to (6), in which

the treatment tool is a pair of retractors, and

the imaging control section estimates an intermediate position between the pair of retractors as the position of the affected area.

(8) The medical observation apparatus according to (6), in which

the treatment tool is an energy treatment tool, and

the imaging control section estimates a front edge position of the energy treatment tool as the position of the affected area.

(9) The medical observation apparatus according to (6), in which

the treatment tool is forceps, and

the imaging control section estimates a front edge position of the forceps as the position of the affected area.

(10) The medical observation apparatus according to any one of (1) to (9), in which

the first toggle operation is an operation performed on an operating device provided in the medical observation apparatus, and

the imaging control section controls the zoom function on a basis of an operation signal output from the operating device in response to the first toggle operation.

(11) The medical observation apparatus according to any one of (1) to (10), in which

the first toggle operation is an operation performed on an operating device external to the medical observation apparatus, and

the imaging control section controls the zoom function on a basis of an operation signal output from the external operating device in response to the first toggle operation.

(12) The medical observation apparatus according to any one of (1) to (11), in which

the first toggle operation is a speech operation performed on a speech input device, and

the imaging control section executes a speech recognition process on a speech signal output from the speech input device, and controls the zoom function on a basis of a recognition result of the first toggle operation.

(13) The medical observation apparatus according to any one of (1) to (12), in which

the imaging control section cancels the electronic zoom on a basis of a second toggle operation that toggles from the mode that controls the electronic zoom to the mode that controls the optical zoom.

(14) The medical observation apparatus according to (13), in which

the second toggle operation is an operation performed on an operating device provided in the medical observation apparatus, and

the imaging control section cancels the electronic zoom on a basis of an operation signal output from the operating device in response to the second toggle operation.

(15) The medical observation apparatus according to (13) or (14), in which

the second toggle operation is an operation performed on an operating device external to the medical observation apparatus, and

the imaging control section cancels the electronic zoom on a basis of an operation signal output from the external operating device in response to the second toggle operation.

(16) The medical observation apparatus according to any one of (13) to (15), in which

the second toggle operation is a speech operation performed on a speech input device, and

the imaging control section executes a speech recognition process on a speech signal output from the speech input device, and cancels the electronic zoom on a basis of a recognition result of the second toggle operation.

(17) The medical observation apparatus according to any one of (1) to (16), including:

an arm including a plurality of links joined to each other by one or a plurality of joint sections; and

the imaging device supported by the arm.

(18) A zoom control method executed by a medical observation apparatus, the method including:

controlling a zoom function of an imaging device on a basis of a first toggle operation that toggles from a mode that controls an optical zoom to a mode that controls an electronic zoom, in which

in the controlling, the zoom function is controlled so that the electronic zoom is executed on a predetermined region in a medical captured image captured by the imaging device, at an optical magnification of an optical system of the imaging device corresponding to when the first toggle operation was detected.

What is claimed is:

1. A medical observation apparatus comprising:
an imaging control section that controls a zoom function of an imaging device on a basis of a first toggle operation that toggles from a mode that controls an optical zoom to a mode that controls an electronic zoom, wherein
the imaging control section controls the zoom function so that the electronic zoom is executed on a predetermined region in a medical captured image captured by the imaging device, at an optical magnification of an optical system of the imaging device corresponding to when the first toggle operation was detected.

2. The medical observation apparatus according to claim 1, wherein
the predetermined region is a region set in advance with respect to the medical captured image.

3. The medical observation apparatus according to claim 2, wherein
the region set in advance is a region centered on a center position of the medical captured image.

4. The medical observation apparatus according to claim 1, wherein
the medical captured image is displayed on a display screen, and
the imaging control section sets the predetermined region on a basis of a gaze position of a detection target of a gaze detection sensor on the display screen from when the first toggle operation was detected.

5. The medical observation apparatus according to claim 1, wherein
the imaging control section
detects a treatment tool from the medical captured image from when the first toggle operation was detected, and
sets the predetermined region on a basis of a position corresponding to the treatment tool in the medical captured image.

6. The medical observation apparatus according to claim 5, wherein
the imaging control section
estimates a position of an affected area relative to the position of the treatment tool in the medical captured image, and
treats the estimated position of the affected area as the position corresponding to the treatment tool.

7. The medical observation apparatus according to claim 6, wherein
the treatment tool is a pair of retractors, and
the imaging control section estimates an intermediate position between the pair of retractors as the position of the affected area.

8. The medical observation apparatus according to claim 6, wherein
the treatment tool is an energy treatment tool, and
the imaging control section estimates a front edge position of the energy treatment tool as the position of the affected area.

9. The medical observation apparatus according to claim 6, wherein
the treatment tool is forceps, and
the imaging control section estimates a front edge position of the forceps as the position of the affected area.

10. The medical observation apparatus according to claim 1, wherein
the first toggle operation is an operation performed on an operating device provided in the medical observation apparatus, and
the imaging control section controls the zoom function on a basis of an operation signal output from the operating device in response to the first toggle operation.

11. The medical observation apparatus according to claim 1, wherein
the first toggle operation is an operation performed on an operating device external to the medical observation apparatus, and
the imaging control section controls the zoom function on a basis of an operation signal output from the external operating device in response to the first toggle operation.

12. The medical observation apparatus according to claim 1, wherein
the first toggle operation is a speech operation performed on a speech input device, and
the imaging control section executes a speech recognition process on a speech signal output from the speech input device, and controls the zoom function on a basis of a recognition result of the first toggle operation.

13. The medical observation apparatus according to claim 1, wherein
the imaging control section cancels the electronic zoom on a basis of a second toggle operation that toggles from the mode that controls the electronic zoom to the mode that controls the optical zoom.

14. The medical observation apparatus according to claim 13, wherein
the second toggle operation is an operation performed on an operating device provided in the medical observation apparatus, and
the imaging control section cancels the electronic zoom on a basis of an operation signal output from the operating device in response to the second toggle operation.

15. The medical observation apparatus according to claim 13, wherein
the second toggle operation is an operation performed on an operating device external to the medical observation apparatus, and
the imaging control section cancels the electronic zoom on a basis of an operation signal output from the external operating device in response to the second toggle operation.

16. The medical observation apparatus according to claim 13, wherein
the second toggle operation is a speech operation performed on a speech input device, and
the imaging control section executes a speech recognition process on a speech signal output from the speech input device, and cancels the electronic zoom on a basis of a recognition result of the second toggle operation.

17. The medical observation apparatus according to claim 1, comprising:
an arm including a plurality of links joined to each other by one or a plurality of joint sections; and
the imaging device supported by the arm.

18. A zoom control method executed by a medical observation apparatus, the method comprising:
controlling a zoom function of an imaging device on a basis of a first toggle operation that toggles from a mode that controls an optical zoom to a mode that controls an electronic zoom, wherein
in the controlling, the zoom function is controlled so that the electronic zoom is executed on a predetermined region in a medical captured image captured by the imaging device, at an optical magnification of an optical system of the imaging device corresponding to when the first toggle operation was detected.
